(19) United States
(12) Patent Application Publication
(10) Pub. No.: US 2006/0184024 A1
(43) Pub. Date: Aug. 17, 2006

(54) Tissue thickness measurement device

(75) Inventors: Luiz B. Da Silva, Danville, CA (US); George Choi, Redwood City, CA (US); Igor Kochemasov, Nighniy Novgorod (RU)

Correspondence Address: John P. Wooldridge, Esq., 114 Honu'ea Pl., Kihei, HI, 96753, US

(73) Assignee: IntelaMetrix, Inc.

(21) Appl. No.: 11/302,039

(22) Filed: Dec. 12, 2005

Related U.S. Application Data
(60) Provisional application No. 60/634,911, filed on Dec. 10, 2004.

(51) Int. Cl. A61B 8/00

(52) U.S. Cl. 600/438

(57) ABSTRACT

A device is described that can be easily used to accurately measure and monitor tissue thickness using ultrasound. The device comprises a remote control and data processing unit, a handheld ultrasound transducer, a display monitor and means for effectively coupling ultrasound to tissue.

This application claims priority to U.S. Provisional Patent Application Ser. No. 60/634,911, titled: “Tissue Thickness Measurement Device,” filed Dec. 10, 2004, incorporated herein by reference.

### BACKGROUND OF THE INVENTION

1. Field of the Invention

The present invention relates to the fields of fitness, healthcare, and cosmetic surgery generally. More particularly, the invention relates to a device and method for measuring soft tissue thickness with a handheld apparatus utilizing ultrasound. This device can be easily employed to monitor changes in adipose and muscle tissue due to changes in fitness or health. The present invention can also be used to measure total body fat by making a plurality of measurements.

2. Description of Related Art

Thickness of tissue layers and in particular adipose (fat) and muscle tissue can be important to evaluate fitness and health. There are a variety of techniques currently used to measure the thickness of the adipose layer. For example skin calipers can be used to measure the thickness of the skin fold produced when the operator pinches a subject's skin. Various equations are used to predict body density and the percent of body adipose tissue (American College of Sports Medicine (ACSM) “Guidelines For Exercise Testing And Prescription”, 53-63 (1995)). However, there are many drawbacks to this form of adipose tissue measurement These measurements are heavily dependent on the operator, and errors and variations frequently occur. Skin fold calipers can only provide an estimate of tissue thickness and are not particularly accurate for tracking small changes.

Another means of determining body density and estimating percent body adipose tissue is a generalized measurement hydrostatic weighing. Hydrostatic weighing requires the subject to be completely immersed in water. This method of measurement could only be employed before and after a liposuction procedure, which would be impractical and costly when the goal is to monitor adipose tissue changes during the surgery. Additionally, the surgeon performing liposculpture and most surgical contouring procedures requires localized measurements. Maintenance of a sterile field is problematic with such a method.

A method and apparatus is needed to efficiently and accurately measure adipose tissue. U.S. Pat. No. 5,941,825 dated October 1996 by Lang et al., recognized that ultrasound could be utilized to conveniently and cost effectively measure layer thickness in an object WO 99/65395 dated December 1999 by Lang et al., builds on the previously referenced patent by using anatomical landmarks to measure changes in body adipose tissue. The aim of these two patents is to measure adipose tissue changes over time as a result of diet and exercise. However, all these patents describe ultrasound transducers that require applying a fluid or gel to get effective acoustic coupling between the transducer and skin. This makes measurements messy and inconvenient for the subject.

There is a need for an accurate, convenient, cost effective means and apparatus to measure tissue thickness accurately. The present invention fulfills this need, and further provides related advantages.

### SUMMARY OF THE INVENTION

It is an object of the present invention is to provide a system for accurately measuring tissue layer thickness to monitor the effects of exercise or diet.

Another object of the present invention is to provide a system to accurately measure percentage body fat and body density.

Another object of the present invention is to provide a system to accurately measure adipose tissue distribution and identify superficial adipose tissue and deep adipose tissue.

These and other objects will be apparent to those skilled in the art based on the teachings herein.

In one embodiment, the present invention comprises a remote control and data processing unit, a handheld ultrasound transducer, a disposable sterile element to acoustically couple the transducer to skin and a monitor to display the information to the user.

The handheld ultrasound transducer uses a single or a plurality of ultrasound generating and detection elements to obtain an effective A-Scan (“Ultrasound in Medicine” Ed. F. A. Duck, A. C. Baker, H. C. Starritt (1997)) of the tissue structure directly below the transducer. The A-scan will show strong reflections at the interface between the various layers i.e., skin, fat, muscle and bone. Strong ultrasound reflections occur at the interfaces due to impedance mismatch between the various materials. The A-scan signal can be analyzed by the control unit to determine the thickness of the various tissue layers (skin, fat, muscle). By making multiple measurements for example, chest, waist and thigh, a percent body fat for the whole body can be calculated. In this mode the device can be used to monitor fitness programs and diet

In one embodiment, the transducer is not connected by a wire or cable to the control unit. The transducer and control unit communicate through a wireless means (e.g., RF communication). The advantage of this is that the control unit and display can be far away from the sterile surgical field. RF communication eliminates having to cover the control unit and cable with sterile bags. In addition, in this embodiment the ultrasound transducer is powered by batteries, which reduce the electrical hazard concern.

The remote control unit acquires the data from the handheld transducer and analyzes the data to produce a table of tissue thickness parameters for all the anatomical points. This data can be displayed in a tabulated list or a color-coded anatomical map that can be easily interpreted by the surgeon. Additionally, the display can show the change in the fat layer thickness during the course of the liposuction procedure. The user can control the display and function of the control unit through a keyboard/mouse interface or touch screen.

Other objects and advantages of the present invention will become apparent from the following description and accompanying drawings.

### BRIEF DESCRIPTION OF THE DRAWINGS

The accompanying drawings, which are incorporated into and form part of this disclosure, illustrate embodiments of the invention and together with the description, serve to explain the principles of the invention.

FIG. 1 shows a sectional view taken through the handheld ultrasound device with a disposable acoustic matching element.

FIG. 2 shows a sectional view taken through the handheld ultrasound device with a water compartment that can release a small amount of water to acoustically couple the ultrasound device to tissue.

FIG. 3 shows a sectional view taken through another embodiment of the handheld ultrasound device that has an integrated level and ruler.

FIG. 4 shows a sectional view taken through another embodiment of the handheld ultrasound device that has an integrated level and ruler.

FIG. 5 shows how the ultrasound measuring device and control unit would be used.

FIG. 6 shows a measured signal using the present invention on a male abdomen.

FIG. 7 shows a measured signal using the present invention on a male bicep.

### DETAILED DESCRIPTION OF THE INVENTION

The object of the present invention is to provide a system for accurately measuring tissue layer thickness. In particular, the system can be used to produce a map of the fat (or adipose) tissue thickness at key anatomical points. These measurements can be monitored and compared to track changes. In one embodiment, the device comprises a remote control and data processing unit, a handheld ultrasound transducer, and a monitor or LCD to display the information to the user.

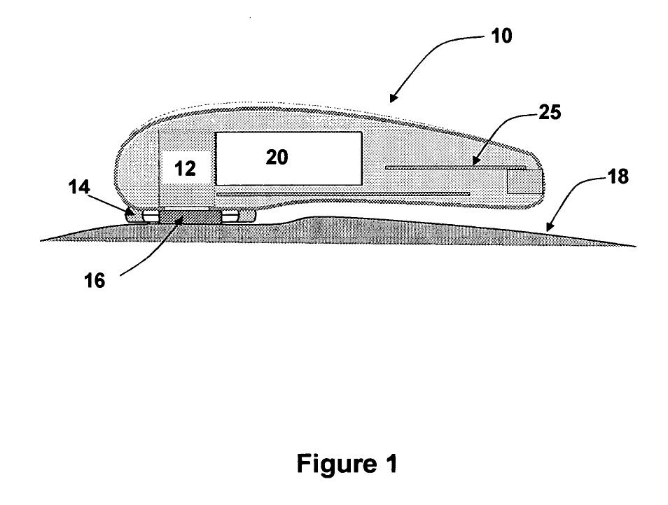

FIG. 1 shows a cross-sectional view of the handheld ultrasound measuring device 10. The device consists of an ultrasound transmitter and receiver 12. The transmitter and receiver can be a single element or two separate elements. The use of two separate elements reduces reflection artifacts and also allows imaging closer to the transmitter element The ultrasound transmitter and detection element can be made of any piezoelectric material. Suitable materials include ceramics (usually lead zirconate titanate (PZT), or plastic (polyvinylidinedifluoride, PVDF). The operating frequency for adequate penetration and resolution in tissue is typically 500 kHz to 10 MHz. For additional information on transducer design and operation refer to “The Physics of Medical Imaging” Ed. Steve Webb (1988) incorporated herein by reference, and “Ultrasound in Medicine” Ed. F. A. Duck, A. C. Baker, H. C. Starritt (1997) incorporated herein by reference. See also U.S. Pat. No. 5,699,806, titled “Ultrasound System With Nonuniform Rotation Corrector” incorporated herein by reference.

In order to efficiently couple the ultrasound energy to the tissue it is important that a matching material is placed between the transducer and the tissue. This can be accomplished by applying a small amount of ultrasound coupling gel to the face of the transducer before applying it to tissue. Alternatively a disposable holder 14 connects to the device 10 to make acoustic contact between the transducer 12 and the matching material 16. The matching material is a high water fraction hydro gel or sol gel similar to that commonly used in electrocardiograms (ECG) electrodes or transcutaneous electric nerve stimulation (TENS) electrodes. The outside surface of the matching material 16 makes contact with the skin 18 and ensures good acoustic contact with minimal reflection at the skin interface. It is important that no air layer exists between the matching material 16 and the skin surface 18. An air layer produces a large reflection and significantly reduces the amount of ultrasound energy that is transmitted into the tissue. U.S. Pat. No. 6,792,301 (Munro et al.), incorporated herein by reference, and references therein describe a suitable material composition.

In order to reduce the risk of contamination a new disposable holder 14 can be used for each customer and visit The use of a solid and adhesive matching material 16 avoids the need to apply acoustic gels or creams to the skin that need to be cleaned off after the procedure.

The device 10 can be powered by a battery 20 or external power cord (not shown). The measured signal can be transferred to a remote computer or microprocessor by wireless means 25 (e.g., Bluetooth, devices conforming to any wireless standard routinely used by computers e.g., IEEE 802.11, acoustic or optical) or cable (not shown). The device 10 can also be powered and also communicate to remote computer by a USB cable.

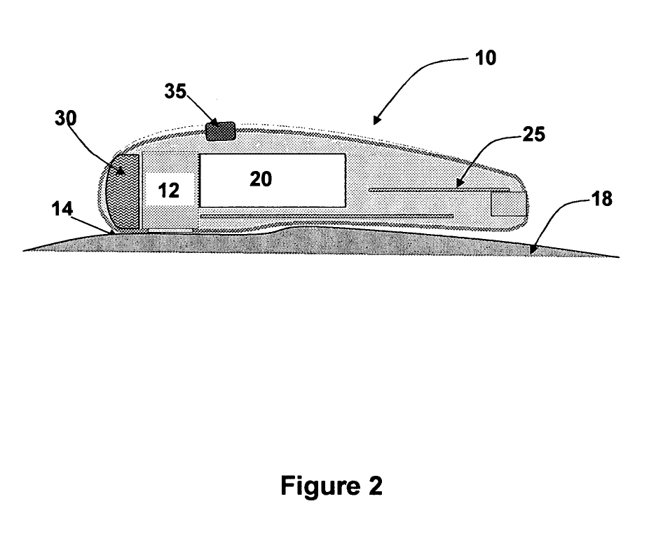

FIG. 2 shows an alternative embodiment of the present invention that contains a refillable water compartment 30. In this embodiment the disposable holder is eliminated and acoustic coupling between the ultrasound transducer 12 and the skin 18 is made by a thin water layer. When making a measurement, the user presses button 35 that causes a small amount of water (1-2 drops) to be released near the surface of the ultrasound transducer 12. The water fills the gap between the transducer 12 and the skin 18 and allows efficient transmission into the tissue. The surface of the ultrasound transducer can be treated to be hydrophilic so that water will easily coat the surface. Instead of water a low viscosity oil or hydrogel could be used.

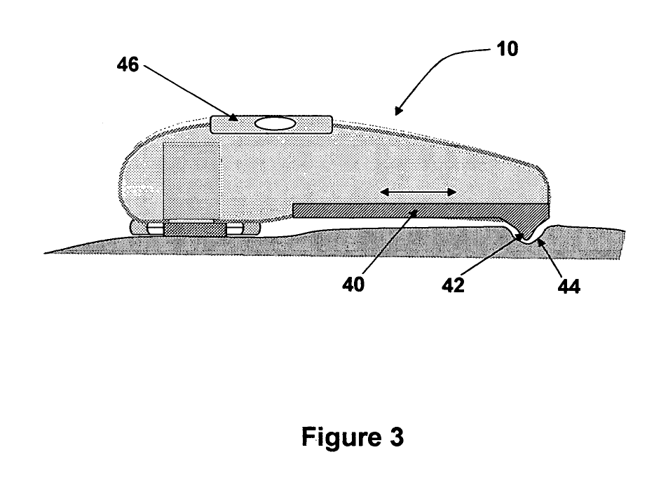

FIG. 3 shows an alternative embodiment of the present invention that contains an integrated ruler 40 (or measuring reference) that can be used to accurately position the transducer relative to a anatomical landmark. The ruler 40 can slide (left and right as shown) to allow the transducer to be placed at the desired distance from the bulbous tip 42. In addition, the device 10 can have an integrated level 46 to further allow the user to accurately set the orientation of the device. The level 46 can be a simple mechanical (e.g. water-bubble) level or an electronic IC based level with LED or LCD display. The ruler and level could be used to consistently make the measurement at the same anatomical position. This is important when monitoring changes over time. For example, by placing the bulbous tip 42 in the umbilicus 44 (belly button) it is possible to consistently make the tissue measurement at the same location.

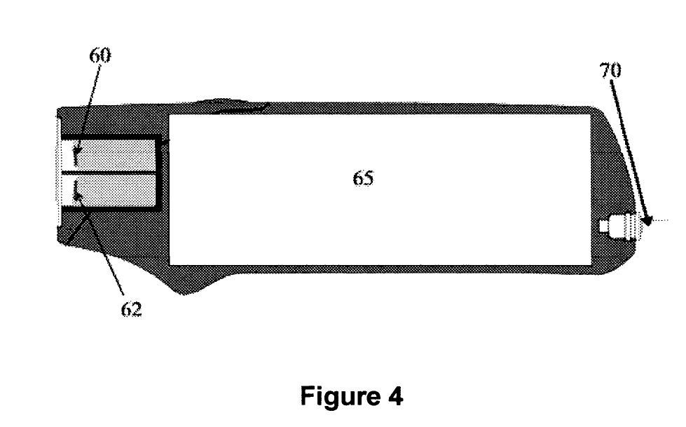

FIG. 4 shows a cross section of an alterative embodiment of the present invention that integrates a separate ultrasound transmitter 60 and receiver 62 into a handheld device. A circuit board 65 drives the transmitter 60 and processes the received signal from the receiver 62 by amplifying it and filtering it before converting it to a digital signal that can be transmitted through the USB cable 70.

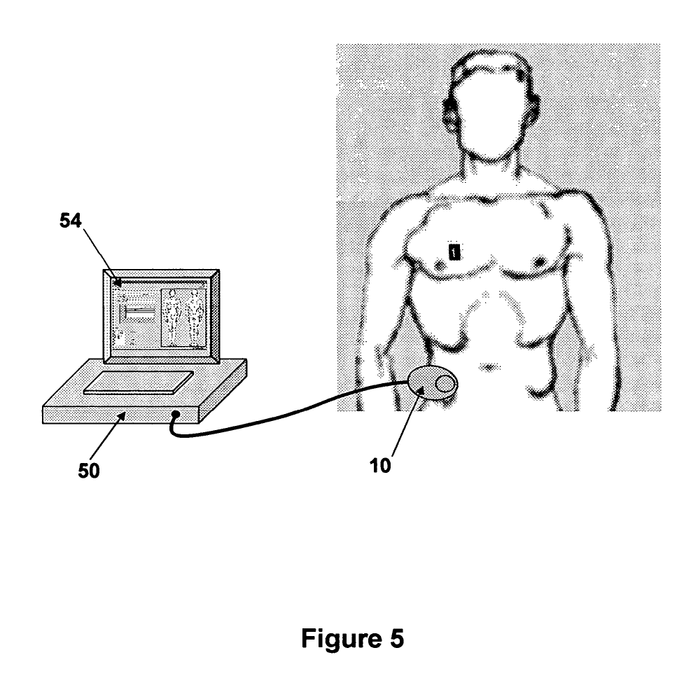

FIG. 5 illustrates how the present invention would be used to measure the local tissue structure. The measuring device 10 is placed on the skin at a point of interest When activated, an ultrasound signal is transmitted into the tissue and the return signal collected. The collected signal is than communicated by cable (as shown) or by wireless means to the remote control unit 50. The control unit 50 displays the recorded waveforms and calculated thickness of relevant layers on a monitor 54. In addition, the control unit 50 stores the waveforms and information about the location of the measurement so that the user can easily monitor changes over time. The control unit can be a portable computer, or PDA (e.g., HP Ipaq, Palm Pilot, etc.) In another embodiment, the device 10 is self contained and a small LCD display on the device 10 displays a summary of each measurement.

The control unit 50 can use user specific data such as age, height, weight and the location of the measurement to improve the signal processing and accurately determine the tissue thickness. Interpreting standard A-scan ultrasound to identify tissue boundaries can be difficult and confusing for untrained users. By using accepted norms as a guide the control unit 50 can accurately determine the adipose tissue thickness.

For the present invention, the operating frequency of the transducer will typically be in the range of 500 kHz to 10 MHz. The higher frequencies have higher spatial resolution but suffer from high tissue attenuation, which limits the thickness of tissue that can be measured. In addition, it is sometimes beneficial to operate the ultrasound transducer at two different frequencies. Since the scattered signal scales strongly with the ultrasound wavelength, the ratio of scattered signal at two frequencies can be used to determined tissue properties.

A curved transducer may be used to provide a weakly focused beam that measures properties over a less than 5 mm diameter region. A small diameter reduces the blurring of layer boundaries due to non-planar layer contours. The transducer is used to both generate the ultrasound pulse and measure the time history of the return acoustic signal. The collected time history signal is a measurement of the back-scattered signal as a function of depth averaged over the ultrasound beam area. The control electronics collect and digitize the signal for further display and analysis. For additional information on transducer design and operation refer to “The Physics of Medical Imaging” Ed. Steve Webb (1988) incorporated herein by reference, and “Ultrasound in Medicine” Ed. F. A. Duck, A. C. Baker, H. C. Starritt (1997) incorporated herein by reference. See also U.S. Pat. No. 5,699,806, titled “Ultrasound System With Nonuniform Rotation Corrector” incorporated herein by reference.

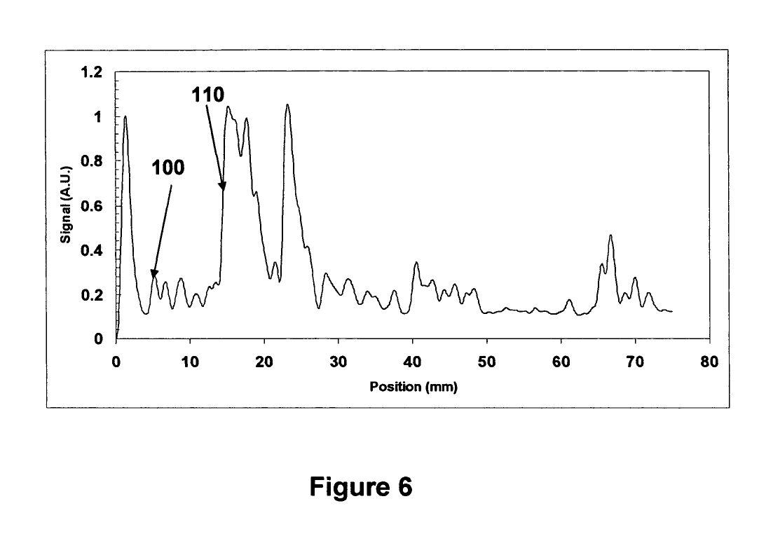

FIG. 6 shows a measured signal using the present invention on a male abdomen. The signal peaks correspond to the interface between the device and skin 100, and; fat and muscle 110. The adipose (fat) layer is located between 100 and 110 and is approximately 9.8 mm thick. Strong ultrasound reflections occur at the interfaces due to impedance mismatch between the various materials. The time history is converted to thickness by the software by using average sound speeds (c). For example, c˜1600 m/s for skin, 1400 m/s for fat, 1600 m/s for muscle, and 3500 m/s for bone (See “Ultrasound in Medicine” Ed. F. A. Duck, A. C. Baker, H. C. Starritt).

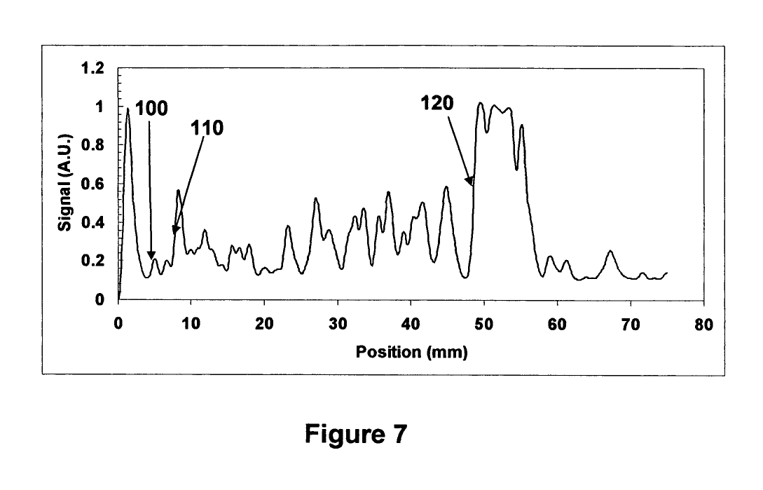

FIG. 7 shows a measured signal using the present invention on a male bicep muscle. The signal peaks correspond to the interface between the device and skin 100, and; fat and muscle 110 and; 120 muscle and bone. The adipose layer is located between 100 and 110 and is approximately 3.2 mm thick. The muscle layer is located between 110 and 120 and is approximately 40.8 mm thick.

In order to accurately detect the interfaces, the control software analyzes the signal and, based on additional input information (e.g., measurement location, client weight, height, age and sex), determines the proper interface position. Strong signals are generally produced at each interface due to large difference in the acoustic impedance of the different tissue types. In addition, muscle tissue generally shows strongly signal fluctuations and that information can be used to distinguish muscle from adipose tissue. Using client weight and height, the body mass index can be calculated and using formulas that relate percentage body fat to body mass index (e.g., Deurenberg P, Yap M, van Staveren W A. “Body mass index and percent body fat A meta analysis among different ethnic groups. Int J Obes Relat Metab Disord 1998; 22:1164-1171, incorporated herein by reference) the approximate thickness of adipose tissue can be calculated. Generally this estimated value can be 25%-50% too high for athletes. So in one version of the algorithm the user can input whether the client has an athletic build or not.

In normal use the measuring device would be applied at a single point or multiple key anatomical points. By making measurements at multiple sites (at least three) you can estimate the body density (D) and the percentage body fat (% BF). For example, by making a measurements at chest, abdomen, and thigh you can estimate the body density (D) and percentage body fat (% BF) with the following equations fro males and females respectively.

For Males: D=1.10938−(0.0008267×sum of chest, abdominal, thigh)+(0.0000016×square of the sum of chest, abdominal, thigh)−(0.0002574×age). Equation is based on a sample of males aged 18-61 Jackson, A. S. & Pollock, M. L. (1978) Generalized equations for predicting body density of men. British J of Nutrition, 40: p 497-504.).

D=1.1043−(0.001327×thigh)−(0.00131×subscapular), based on a sample aged 18-26. Sloan A W: Estimation of body fat in young men., J Appl. Physiol. (1967);23:p 311-315.

% BF=(0.1051×sum of triceps, subscapular, supraspinale, abdominal, thigh, calf)+2.585, based on a sample of college students. Yuhasz, M. S.: Physical Fitness Manual, London Ontario, University of Western Ontario, (1974).

For Females: D=1.0994921−(0.0009929×sum of triceps, suprailiac, thigh)+(0.0000023×square of the sum of triceps, suprailiac, thigh)−(0.0001392×age), based on a sample aged 18-55. Jackson, et al. (1980) Generalized equations for predicting body density of women. Medicine and Science in Sports and Exercise, 12:p 175-182.

D=1.0764−(0.0008×iliac crest)−(0.00088×tricep), based on a sample aged 17-25. Sloan, A. W., Burt A. J., Blyth C. S.: Estimating body fat in young women., J. Appl. Physiol. (1962);17:p 967-970.

% BF=(0.1548×sum of triceps, subscpular, supraspinale, abdominal, thigh, calf)+3.580, based on a sample of college students. Yuhasz, M. S.: Physical. Fitness Manual, London Ontario,University of Western Ontario, (1974).

Although these equations refer to thickness measurements taken with calipers they can also be applied when fat thickness measurements are made with our more accurate device. In addition, a wide variety of other equations exist that offer greater accuracy but in some require additional information (e.g. accurate age, body type).

Software within the control unit can guide the user through the process of collecting measurements at the key anatomical sites and then display the calculated % body fat (% BF) and Body Density (D).

This device can also be used by plastic surgeons to track and monitor liposuction procedures.

Another application of this ultrasound device is for measuring the different adipose tissue layers in the abdominal region. In particular, superficial and deep adipose tissue form two separate regions around the abdomen. Deep adipose tissue thickness is an important indicator of heath risk.

The above descriptions and illustrations are only by way of example and are not to be taken as limiting the invention in any manner. One skilled in the art can substitute known equivalents for the structures and means described. The full scope and definition of the invention, therefore, is set forth in the following claims.

We claim:

1. A method, comprising:
applying at least one ultrasound transducer to the surface of a skin portion of a subject under test;
transmitting ultrasound pulses from said transducer into said skin portion, wherein interfaces between layers beneath said skin portion will reflect a portion of said ultrasound pulses to produce a return signal;
detecting said return signal; and
calculating from said return signal the location of at least one tissue boundary by using at least one parameter that is specific to said subject under test, wherein said parameter is selected from the group consisting of age, height, weight, sex, and location of said skin portion.

2. The method of claim 1, wherein said at least one tissue boundary comprises an interface between adipose tissue and muscle.

3. The method of claim 1, wherein said at least one tissue boundary comprises an interface between muscle and bone.

4. The method of claim 1, wherein said return signal is further analyzed to determine the thickness of at least one tissue layer beneath said skin portion.

5. The method of claim 4, wherein said at least one tissue layer comprises a fat layer, wherein the steps of applying, transmitting, detecting and calculating are repeated at different locations on said subject under test to produce a plurality of return signals, the method further comprising calculating a percentage of body fat of said subject under test by using said plurality of return signals.

6. The method of claim 5, further comprising producing a map of fat thickness.

7. The method of claim 1, further comprising calculating the body mass index of said subject from the weight and height of said subject

8. The method of claim 7, wherein said return signal is further analyzed to determine the thickness of at least one tissue layer beneath said skin portion, wherein said at least one tissue layer comprises a fat layer, wherein the steps of applying, transmitting, detecting and calculating are repeated at different locations on said subject under test to produce a plurality of return signals, the method further comprising calculating a percentage of body fat of said subject under test by using said plurality of return signals, the method further comprising approximating adipose tissue thickness by relating said percentage body fat to said body mass index.

9. An apparatus, comprising:
an ultrasound transmitter and receiver in a handholdable housing, wherein ultrasound pulses from said transducer can be transmitted into a skin portion of a subject under test, wherein interfaces between layers beneath said skin portion will reflect a portion of said ultrasound pulses to produce a return signal, wherein said receiver can detect said return signal;
means for powering said ultrasound transmitter and receiver;
a computer system comprising hardware and software, wherein said software comprises means for calculating from said return signal the location of at least one tissue boundary by using at least one parameter that is specific to said subject under test, wherein said parameter is selected from the group consisting of age, height, weight, sex, and location of said skin portion; and
means for transmitting signals from said ultrasound transmitter and receiver to said computer system.

10. The apparatus of claim 9, wherein said ultrasound transmitter and receiver are a single element.

11. The apparatus of claim 9, wherein said ultrasound transmitter and said receiver are two separate elements.

12. The apparatus of claim 9, further comprising means for coupling said ultrasound transmitter and receiver to said skin portion.

13. The apparatus of claim 12, wherein said means for coupling comprises a disposable ultrasound coupling gel holder.

14. The apparatus of claim 12, wherein said means for coupling comprises a refillable water compartment.

15. The apparatus of claim 14, wherein said ultrasound transducer comprises a hydrophilic surface.

16. The apparatus of claim 9, further comprising a ruler integrated onto said handholdable housing.

17. The apparatus of claim 9, further comprising an level integrated onto said handholdable housing.

18. The apparatus of claim 9, wherein said transducer comprises a curved surface configured to provide a weakly focused beam.
